(19) United States
(12) Patent Application Publication
(10) Pub. No.: US 2004/0133933 A1
(43) Pub. Date: Jul. 8, 2004

(54) Medane genes and proteins

(76) Inventors: Jordi Guimera-Vilaro, Dachau (DE); Wolfgang Wurst, Munchen (DE); Daniela Vogt-Weisenhorn, Munchen (DE); Laure Bally-Cuif, Munchen (DE)

Correspondence Address: JENKINS & WILSON, PA, 3100 TOWER BLVD, SUITE 1400, DURHAM, NC, 27707, US

(21) Appl. No.: 10/631,550

(22) Filed: Jul. 31, 2003

Related U.S. Application Data
(63) Continuation of application No. PCT/EP02/01077, filed on Feb. 1, 2002.

(30) Foreign Application Priority Data
Feb. 1, 2001 (DE) 10104584.0

(51) Int. Cl. A01K 67/00; C07H 21/04; C12N 15/85

(52) U.S. Cl. 800/8; 536/23.2; 435/455

(57) ABSTRACT

This invention relates to a novel DNA sequence encoding a bHLH transcription factor in vertebrates, preferably mammals, e.g. in mice or humans, as well as the expressed transcription factor. The invention further relates to vectors containing said DNA sequences and host cells transformed by these vectors. Furthermore, the invention encompasses antibodies specific for the transcription factors as well as the use of the DNA sequences and transcription factors in the diagnosis or therapy of neurodegenerative diseases, e.g. Parkinson's disease. The present invention further relates to an ex vivo method of producing dopaminergic cells and the therapeutic use of these dopaminergic cells.

### RELATED APPLICATIONS

This application is a continuation of PCT patent application number PCT/EP02/01077, filed Feb. 1, 2002, which claims priority to German patent application number 10104584.0, filed Feb. 1, 2001, the disclosures of each of which are incorporated herein by reference in their entirety.

### TECHNICAL FIELD

This invention relates to a novel DNA sequence encoding a bHLH transcription factor in vertebrates, preferably mammals, e.g. in mice or humans, as well as the expressed transcription factor. The invention further relates to vectors containing said DNA sequences and host cells transformed by these vectors. Furthermore, the invention encompasses antibodies specific for the transcription factors as well as the use of the DNA sequences and transcription factors in the diagnosis or therapy of neurodegenerative diseases, e.g. Parkinson's disease. The present invention further relates to an ex vivo method of producing dopaminergic cells and the therapeutic use of these dopaminergic cells.

### BACKGROUND ART

Up to now, a variety of DNA sequences has been identified, which code for vertebrate bHLH transcription factors. For example, approximately 140 human bHLH transcription factors have been identified based on the human genome sequences. However, the knowledge is limited to the sequence data per se and no function of these transcription factors has been elucidated.

Neurotransmitters are endogenous substances that are released from neurons and produce a functional change in the properties of the target cells. The amino acid tyrosine is the precursor for three different amine neurotransmitters that contain a chemical structure called a catechol. These neurotransmitters are collectively called catecholamines and include dopamine, norepinephrine, and epinephrine. The catecholamine neurotransmitters contain the enzyme tyrosine hydroxylase (TH), which catalyses the initial steps in catecholamine biosynthesis (Nagatsu et al., 1964) the conversion of the amino acid L-tyrosine into a compound called L-dopa (3,4-dihydroxyphenylalanine).

The Catecholaminergic system is one of the major mono-aminergic systems in the brain stem. Catecholamines neurons have been shown to be in regions of the nervous system involved in the regulation of movement, mood, attention, and visceral function and are composed of dopamine, noradrenaline and adrenaline producing neurons (reviewed in Smeets and Reiner, 1994).

The dopaminergic system is highly organised topographically. In mammals, the dopaminergic neurons (DA) reside in the telencephalon, diencephalon and midbrain. DA neurons of the adult mammal have been placed into nine distinct groups (Specht et al., 1981; Bjorklund and Lindvail, 1984; Voorn et al., 1988). The telencephalon contains two smaller groups of DA neurons restricted to the olfactory bulb (group 16) and the retina (group A17).

The most prominent groups are the so-called mesencephalic dopaminergic neurons (mesDA) residing in the ventral midbrain (groups A8, A9, A10) and in the diencephalon groups A11-A15 involved in the release of pituitary hormones.

The mesDA are a limited set of neurons and can be subdivided into three groups, the ventral tegmental area innervate the neocortex (group A10), the substantia nigra pars compacta innervate the striatum (group A9) and the retrorubral nucleus (group A8). In the mouse, the generation of these DA cells can be monitored from approximately embryonic day 11.5 (E11.5) by expression of TH.

The mammalian DA neurons regulate behaviour and voluntary movement control, reward-associated behaviour, and hormonal homeostasis and has been implicated in psychiatric and affective disorders (Grace et al., 1997). The selective degeneration of dopaminergic neurons within the mesDA system is the direct cause of the motor disorder characteristic of Parkinson's disease (Jellinger, 1973; Forno, 1992; Golbe, 1993). Whereas overstimulation of ventral tegmental DA neurons has been implicated in affective disorders like manic depression and schizophrenia (Ritz et al., 1987) and in behavioural reinforcement and drug addiction (Seeman et al., 1993).

Generation of cellular diversity in the developing mammalian brain involves cascades of secreted signalling molecules that acts in the specification of the distinct neuronal cell types. This coarse-grained pattern is subsequently reinforced and refined by diverse, locally acting mechanisms resulting in a precise regional variation in cell identity (Lumsden et al., 1996). The network by which mesDA neurons assume their specific identity and are confined to the ventral part of the midbrain in mouse embryos is poorly understood. The progenitors for this neuronal cell type lie on the ventral part of the midbrain as early as E9 and they differentiate in this region at a time between E9 and E14 (Hynes and Rosenthal, 1999). Their specification is dependent on multiple co-operating epigenetic signals like the presence of ventrally expressed Sonic Hedgehog (Shh), a secreted protein important for ventral cell fates in the central nervous system (CNS) (Echelard et al., 1993; Ericson et al., 1995). Another secreted protein from the mid-/hindbrain (MHB) boundary important for the dopaminergic phenotype is Fibroblast growth factor-8 (FGF8). A combined signalling of these two secreted proteins has been shown to mediate the generation of dopamine progenitors cells (Ye et al., 1998), but finally they are specified in response to yet unidentified inductive intracellular signal. Although some of these intracellular signals, the homeobox gene Ptx3 (Smidt et al., 1997) and the orphan nuclear hormone receptor Nurrl (Castillo et al., 1999), have been shown linked to the TH pathway, none of these early signals explain the process underlying the specification of DA neurons, (Hynes and Rosenthal 1999).

Thus, the intracellular transcription factor required specifying induction of midbrain-dopamine cell lineage remained to be identified.

The understanding of the basis mechanisms of vertebrate cell differentiation has been greatly advanced by the findings of transcription factors such as the basic helix-loop-helix (bHLH) (Lee, 1997). The bHLH proteins comprise evolutionarily ancient transcription factors united by conservation solely within the bHLH domain (Murre et al., 1994). bHLH proteins have been found to function as transcriptional regulators in a variety of developmental processes (Olson, 1990, Cabrera and Alonso 1991, Van Doren et al., 1992, Martinez et al., 1993), regulating the determination of neural progenitor cells (Campos-Ortega, 1993) and other cell fate decisions (Carmena et al., 1995, Corbin et al., 1991, Ruohola et al., 1991, Xu et al., 1992).

A special class of bHLH proteins is defined by the translational products of the Drosophila genes hairy (Rushlow et al., 1989) and the E (spl) (Kläambt et al., 1989; Knust et al., 1992; Delidakis and Artavanis-Tsakonas, 1992). In Drosophila, hairy-related bHLH factors are involved in delimiting expression territories and/or domains of cell specification within the embryo and larva, controlling cell fate specification choices during multiple developmental processes, including neurogenesis and myogenesis, where E (spl) factors includes 7 clustered small bHLH proteins comprising the majority of direct transcriptional targets of Delta/Notch signalling (Fischer and Caudy, 1998).

During development, many cell type specifications in higher animals are controlled by intercellular communication governed by the Notch signalling pathway, a gene that controls cellular differentiation, establishment of sharp boundaries of gene expression and generation of cell-type diversity (Artavanis-Tsakonas et al., 1995). In Drosophila, Notch target genes include the E (Spl) genes (Jennings et al., 1994; de Celis et al., 1996) and the Notch signalling pathway is shown to be conserved in mammalian neurogenesis (de la Pompa et al., 1997), including HES-1 gene (Jarriault et al., 1995).

There have been suggestions that also Mammalian homologues of Drosophila bHLH are playing an important role as regulators of cell fate decisions in the developing nervous system and inducers of neuronal differentiation at the level of gene transcription. (Reviewed by Jan and Jan, 1993; Lee, 1997) However, no specific function of such a bHLH transcription factor in the developing nervous system has been identified yet.

### SUMMARY OF THE INVENTION

Therefore, it is the object of the present invention to provide new bHLH transcription factors, which are involved in and can be used for the specification of cells in the developing nervous system, in particular in the induction of dopaminergic neurons.

Herein disclosed are the vertebrate Medane genes, which are novel basic-helix-loop-helix (bHLH) genes involved in the specification and differentiation of neural cells, in particular in the induction of dopaminergic neurons.

The DNA sequences of the invention encode bHLH transcription factors which show some sequence homology to the previously described hairy and Enhancer of split [E (spl)] genes of Drosophila. Therefore, this DNA sequence is generally termed Medane (for Mesencephalic Dopaminergic neurons E (spl) and hairy related gene).

Surprisingly, it turned out that Medane was capable of specifically inducing neurotransmitter secreting cells in vertebrates. Unexpectedly, the inventors found out that the development of dopaminergic neurons could be induced by ectopic expression of Medane in vertebrates.

Therefore, this invention preferably finds application for the substitution of degenerated or lost dopaminergic neurons in vertebrates. Thus, the present invention provides a new therapy, by which the drawbacks of the prior art therapies, i.e. the transplantation of embryonic mesencephalic cells, can be avoided. Following the prior art transplantations, these cells showed a poor survival rate and dopamine production in the treated patients.

As used herein, the term specification or determination means the commitment of a cell to a particular path of differentiation, even though there may be no morphological features that reveal this determination (is not yet expressing the characteristic phenotype). The term differentiation means a process in the development of a multicellular organism by which cells become specialized for particular function.

The DNA sequences of mouse and human Medane genes according to the present invention are disclosed in Seq. ID. No. 3 and 4 for the genomic DNA sequence and in Seq. ID. No. 1 and 2 for the cDNA sequence.

This invention is directed to said Medane genes, fragments thereof and the related cDNA which are useful, for example, as follows: 1) to produce transcription factors by biochemical engineering; 2) to prepare nucleic acid probes to test for the presence of the Medane gene in cells of a subject: 3) to prepare appropriate polymerase chain reaction (PCR) primers for use, for example, in PCR-based assays or to produce nucleic acid probes; 4) to identify Medane transcription factors as well as homologues or near homologues thereto; 5) to identify various mRNAs transcribed from Medane genes in various tissues and cell lines, preferably human; and 6) to identify mutations in Medane genes.

The invention further concerns the hitherto unknown mammalian transcription factors, encoded by the Medane gene.

The Medane gene encodes a protein of 241 amino acids and functions in the nucleus as determined by cytogenetic studies. Medane is specifically expressed in the precursors of dopaminergic neurons and its expression starting, for example, at mouse embryonic day 9 (E9), is confined to the ventral part of the developing mouse midbrain, where mesencephalic dopaminergic neurons (mesDA) appear later on. In situ hybridisation studies show a spatio-temporal correlation between Medane and Tyrosine hydroxilase (TH) expression along mouse catecholaminergic neurons development, and show that Medane is expressed in the precursor cells that will give rise to this neuronal cell lineage. Moreover, ectopic expression of Medane in vivo by electroporation of zebrafish (Danio rerio) embryos shows specification and differentiation of new clusters of TH positive cells. Taken together, these results indicate that Medane is a unique bHLH and the earliest transcription factor marking the mesDA neurons, and is involved in developmental determination and early commitment of mesDA neuronal lineage.

It will be understood that the practice of the invention is not limited to the use of the exact DNA sequence as defined in Seq. ID. No. 1-4. Modifications to the sequences, such as deletions, insertions, or substitutions in the sequence which produce silent changes in the resulting protein molecule are also contemplated.

For example, alterations in the gene sequence which result in the production of a chemically equivalent amino acid at a given site are contemplated. Preferably, amino acid substitutions are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Nucleotide changes which result in alteration of the N-terminal and C-terminal portions of the protein molecule frequently do not alter protein activity, as these regions are usually not involved in biological activity. It may also be desirable to eliminate one or more of the cysteines present in the sequence, as the presence of cysteines may result in the undesirable formation of multimers when the protein is produced recombinantly, thereby complicating the purification and crystallization processes. Each of the proposed modifications is well within the routine skill in the art, as is determination of retention of biological activity of the encoded products.

Therefore, where the phrase “DNA sequence” is used in either the specification or the claims, it will be understood to encompass all such modifications and variations which result in the production of a biologically equivalent Medane protein, i.e. a bHLH transcription factor. In particular, the invention contemplates those DNA sequences which are sufficiently duplicative of the sequences disclosed so as to permit hybridization therewith under standard high stringency southern hybridization conditions, such as those described in Maniatis et al. (Molecular Cloning. A Laboratory Manual. Cold Spring Harbor Laboratory, 1982).

Furthermore, said variants also comprise nucleic acid changes due to the degeneracy of the genetic code, which code for the same or functionally equivalent transcription factor as the nucleic acids specifically defined herein.

According to a further aspect, the present invention is directed to a purified isolated mouse/human transcription factor for the induction of dopaminergic neurons comprising the amino acid of Seq. ID. No. 5/6 and homologues or fragments thereof which retain biological activity.

Short specific protein domains can be used to internalize proteins with a specific function into live cells across the blood-brain barrier (1-6). This signal peptide sequence is necessary and sufficient for nuclear internalisation, and can be used as a importing vehicle for cellular import of exogenous proteins when fused to them (7-8).

Therefore, the present invention is also directed to fusion proteins, comprising the herein described transcription factor as well as a signal peptide, which allows the delivery of said transcription factor into a target cell. For example, the TAT sequence can be used fere, which triggers the internalization of genetically fused proteins into the nucleus in a high efficiency manner (2-4; 11). It was reported that TAT-beta-galactosidase fusion protein was transduced rapidly into cells, reaching near maximum intracellular concentrations in less than 15 min (4).

Therefore, the present invention also provides for a noninvasive intracellular way to deliver the functional properties of Mdn protein into target cells and/or tissues, therefore mediating cell fate decisions, according to the functional properties of the Mdn protein described in this patent.

A Tat sequence can be added at the N- or C-terminus of the Mdn protein to mediate out-side-in protein importation. A Histidine taq sequence (His×6) can also be introduced to purify the protein. An epitope tag could also be included in order to detect and/or follow the imported fusion protein in targeted cells using a specific antiepitope antibody. The recombinant protein might then e.g. be expressed in an insect cell line, mediated by the baculovirus expression system (Life technologies), then purified and tested for biological properties.

In vivo and/or in vitro protein transduction of such a biological active fusion protein (Mdn-signal peptide) results in delivery of the biologically active Mdn-protein properties in tissue or cells. The translocation of such bioactive Mdn fusion-protein into the nucleus of the targeted cells or tissue, can influence nuclear activity which could induce an appropriate transcriptional response in order to activate signal transduction pathways to conduct Dopaminergic-cell fate specification and differentiation, conducted by the functional properties of Mdn protein described herein. To achieve this physiological implications, Mdn-signal peptide fusion-protein can be delivered either in vitro (in preparation for tissue or cell transplantation) or in vivo (for specific neuronal identity regeneration) mediated by injection into the lateral ventricles of the central nervous system of the patient, or by intraperitoneal injection. In post of the intraperitoneal claim, recent studies in mice supports the idea that intraperitoneal injection of large proteins fused to the protein transduction domain of the human immunodeficiency virus TAT protein results in delivery of the biologically active fusion protein to all tissues in mice, including the brain (12).

Taken into account altogether, the invention described here allows direct internalization of exogenous Mdn-signal peptide fusion protein by intact live cells into living organism (patients) or into cultured cells, in bulk concentration, in the context of protein therapy, as well as for functional studies with model organisms, given the functional properties of Mdn protein, the low toxicity of this type of protein delivery and the high efficiency of internalization by all of the cells.

The invention further relates to the biochemical engineering of the Medane genes, fragments thereof or related cDNA.

For example, said gene or a fragment thereof or related cDNA can be inserted into a suitable expression vector. The host cells can be transformed with such an expression vector and an Medane transcription factor is expressed therein. Such a recombinant protein or polypeptide can be glycosylated or nonglycosylated, preferably glycosylated, and can be purified to substantial purity. However, it is possible to produce proteins which are synthetically or otherwise biologically prepared.

Numerous vectors suitable for use in transforming bacterial cells are well known. For example, plasmids and bacteriophages, such as lambda phage, are the most commonly used vectors for bacterial hosts, and for E. coli in particular. In both mammalian and insect cells, virus vectors are frequently used to obtain expression of exogenous DNA. In particular mammalian cells are commonly transformed with SV40 or polyoma virus; and insect cells in culture may be transformed with baculovirus expression vectors. Yeast vector systems include yeast centromere plasmids, yeast episomal plasmids and yeast integrating plasmids.

Alternatively, the transformation of the host cells can be achieved directly by naked DNA without the use of a vector. Production of Medane by either eukaryotic cells or prokaryotic cells is contemplated by the present invention. Examples of suitable eukaryotic cells include vertebrate cells, plant cells, yeast cells and insect cells. Preferably, mammalian stem cells are used. A far as human cells are concerned, embryonic and adult stem cells are preferred. Suitable prokaryotic hosts, in addition to E. coli, include Bacillus subtilis.

The invention also relates to a method for producing a transcription factor/polypeptide comprising growing a culture of the cells of the invention in a suitable culture medium, and purifying the protein from the culture.

The bHLH transcription factors of this invention are serologically active, immunogenic and/or antigenic. They can further be used as immunogens to produce specific antibodies, polyclonal and/or monoclonal.

These specific antibodies can be used diagnostically/prognostically and may be used therapeutically. Medane specific antibodies can be used, for example, in laboratory diagnostics, using immunofluorescence microscopy or immunohistochemical staining, as a component in immunoassays for detecting and/or quantitating Medane antigen in, for example, clinical samples, as probes for immunoblotting to detect Medane antigen, in immunoelectron microscopy with colloid gold beads for localization of Medane proteins/polypeptides in cells, and in genetic engineering for cloning the Medane gene or fragments thereof, or related cDNA. Such specific antibodies can be used as components of diagnostic/prognostic kits, for example, for in vitro use on histological sections. Still further, such antibodies can be used to affinity purify Medane proteins and polypeptides.

The invention further relates to a composition comprising a hybridoma which produces a monoclonal antibody having binding specificity to any one of the disclosed transcription factors.

Antibodies are normally synthesized by lymphoid cells derived from B lymphocytes of bone marrow cells. Lymphocytes derived from the same clone produce immunoglobulin of a single amino acid sequence. Lymphocytes cannot be directly cultured over long periods of time to produce substantial amounts of their specific antibody. However, Kohler et al., 1975, Nature, 256:495, demonstrated that a process of somatic cell fusion, specifically between a lymphocyte and a myeloma cell, could yield hybrid cells (“hybridomas”) which grow in culture and produce a specific antibody called a “monoclonal antibody”. Myeloma cells are lymphocyte tumour cells which, depending upon the cell strain, frequently produce an antibody themselves, although “non-producing” strains are known.

The invention further relates to a recombinant non-human mammalian in which the DNA sequence of claim 1 has been inactivated. Preferably, a recombinant mouse is provided, in which the DNA sequence of Seq. ID. No. 5 has been inactivated. Thus, an animal model may be established using such a recombinant knock-out mouse. These animal models allow further insights in the aetiology of several disorders in connection with degeneration of neural cells.

The invention still further concerns nucleic acid probes that are substantially complementary to nucleic acid sequences of the Medane genes. Preferred nucleic acid probes of this invention are those with sequences substantially complementary to the sequences of claims 1-9. The term “probes” includes naturally occurring or recombinant or chemically synthesized single- or doublestranded nucleic acids. They may be labelled by nick translation, Klenow filling reaction, PCR or other methods well known in the art. The preparation and/or labelling of the probes presented in the invention is described in Sambrook, J. et al., 1989, Molecular Cloning: A Laboratory Manual, Cold Spring Harbor Laboratory, NY; or Ausubel, F. M. et al., 1989, Current Protocols in Molecular Biology, John Wiley & Sons, New York N.Y., both of which are incorporated herein by reference in their entirety.

Test kits of this invention can comprise such probes which are useful diagnostically/prognostically for neurodegenerative diseases. Preferred test kits comprise means for detecting or measuring the hybridisation of said probes to the Medane gene or to the mRNA product of the Medane gene, such as a visualizing means.

Immunoassays can be embodied in test kits which comprise Medane proteins/polypeptides and/or Medane-specific antibodies. Such test kits can be in solid phase formats, but are not limited thereto, and can also be in liquid phase format, and can be based on ELISAS, particle assays, radiometric or fluorometric assays either unamplified or amplified, using, for example, avidin/biotin technology.

As such, the Medane transcription factors are useful both in vivo and in vitro, in growth, maintenance and regeneration of nerve cells of the central nervous system, especially in dopaminergic precursor cells.

According to a preferred emodiment, the present invention comprises an ex vivo method of producing dopaminergic neurons, which comprises the following steps: providing neural embryonic stem cells, neural adult stem cells and/or embryonic stem cells; contacting said cells with an effective amount of the transcription factor of the present invention; culturing said cells under conditions, which allow the specification and differentiation to dopaminergic neurons; and recovering the mature dopaminergic neurons. Furthermore, the present invention encompasses dopaminergic neurons, which are obtainable by this ex vivo method. These neurons may also be present in a composition, which comprises an effective amount of the dopaminergic neurons in combination with a pharmaceutically acceptable carrier.

In view of the evident role in differentiation, the Medane protein can also be used as a regeneration factor. In particular, Medane may be useful in the treatment of neurodegenerative diseases, for example Parkinson's disease. However, the term “neurodegenerative diseases” as used herein also encompasses all other diseases in which the dopaminergic system is involved, p.e. affective disorders like manic depression and schizophrenia and in behavioural reinforcement and drug addiction.

The Medane DNA gene and protein is useful in the treatment of progenitor cells, e.g. stem cells, to promote the differentiation of these cells to mature neural cells, in particular dopaminergic neural cells. In general, human embryonic stem cells are useful for the ex vivo culturing of neural cells, which then are administered to a patient suffering from a neurodegenerative disease.

Alternatively, adult stem cells isolated from the patient to be treated, may be differentiated ex vivo to fully developed neural cells and then returned to the patient for the substitution of degenerated or lost neural cells.

Thus, an in vivo administration of Medane is significantly simplified by the discovery of the gene sequence, particularly in treatment of central nervous system injury.

The identification of the gene and its sequence permits construction of transgenic cells such as fibroblasts, monocytes, or macrophages, which may be engineered to permit expression of the Medane gene and used as an implant for treatment of neurodegenerative disorders, or any conditions in which enhancement of nerve cell growth and/or regeneration would be desirable.

Moreover, the therapeutic use of the Medane transcription factor is not limited to treatment of humans alone. In fact, in view of the conserved nature of this protein among distantly related species, administration of Medane in any form may be beneficial for veterinary application as well. Therapeutic compositions comprise Medane in an amount effective to induce the desired biological activity in combination with a pharmaceutically acceptable liquid or solid carrier. Alternately, the composition may comprise a pharmaceutically acceptable aggregation of compatible transgenic cells capable of expressing Medane in vitro, as an implant for central nervous system regeneration or differentiation treatment.

As used herein, the abbreviation “MDN/MDN” is related to human DNA and amino acid sequences, respectively; the abbreviation “Mdn/Mdn” to mouse DNA and amino acid sequences, respectively.

### BRIEF DESCRIPTION OF THE DRAWINGS

FIG. 1 Genomic organisation of Mdn/MDN genes. The exons and introns are numbered and drawn to scale. Open boxes are noncoding regions; black boxes represent the translated region. The bHLH domain and the Orange domain are depicted, as well as the bipartite nuclear translocation signal and proline rich region.

FIG. 2 Alignment of bHLH domains of hairy/E (spl)-related genes and Mdn. Alignment was generated using the Vector NTI Package programs. Bold letters depicts amino acid residues conserved among at least four bHLH proteins. Grey letters depicts similar amino acid residues. A dash indicates spacing between amino acids to achieve best alignment. Accession numbers for: Hesl (gi 475014); Hes3 (gi 7594823); Her6 (gi 1279398); Her3 (gi 1279394); Her8b (gi 10863869); hes2 (gi 6680207); hes3 (gi 6680209); Hes4 (gi 11423215); hes5 (gi 6754182); SHARP1 (gi 2267587); E (spl) m7 (gi 85074); hairy (gi 85137); DPN (gi 3913501); DEC1 (gi 11414986); HEY1 (gi 7018332); Her1 (gi 10880827); Her2 (gi 1279392); Her4 (gi 1279396); her7 (gi 7576909); her5 (X95301).

FIG. 3

Subcellular localisation of Mdn. Fluorescent image of transfected human embryonic kidney 293 cells with 0.5 and 2 μg of EGFP-N1 blank vector (a+b, respectively). 0.5 and 2 μg Mdn-EGFP-N1 fusion protein (c+d, respectively). The same cells as above visualized with fluorescent light combined with phase-contrast (e). A dashed white line is delimiting the periphery of the cell.

FIG. 4

Tissue distribution of Mdn/MND transcripts. a) The Mdn mRNA was detected only in testis tissue when an 844 bp partial cDNA was used to probe a mouse adult northern blot. b) Mouse foetal Northern blot showing starting expression of Mdn as early as E8.75. We note a pick of expression at E11 in the ventral part of the mesencephalon. Longer exposures did not reveal other bands. c) The MDN mRNA was not detected in any tested tissue. Equal loading of mRNAs was checked as shown in the figure.

FIG. 5

Whole mount in situ hybridisation of Mdn. Mouse E9.5 embryo showing the pattern of expression of Mdn restricted to the developing ventral mesencephalon anterior to the isthmic organiser.

FIG. 6

Expression of Medane and TH at in horizontal sections of E12.5 embryos. At E12.5 Medane is strongly expressed in specific regions of the ventricular zone. It is most prominent in the dorsal part of the mesencephalic vesicle, the substantia nigra (sn; 1, 2), the hypothalamus (hyp; 3, 4), locus coeruleus (LC, 4, 5), in the ganglionic eminences, the future striatum (str; 5, 6) and also in the spinal cord (sc; 6). Parallel sections hybridised with a probe recognizing TH (1″-6″) revealed, that TH is expressed in regions close to Medane expressing domains.

TH-expressing cells are located further away from the ventricular zone, than Medane expressing cells. This expression pattern suggests that Medane might be expressed in cells in the ventricular zone destined to become TH-expressing cells. Also the stronger expression of Medane in the substantia nigra as compared to the still relative weak expression of TH suggests that Medane might be expressed before TH in this region. An additional expression domain of TH, which does not show Medane expression at this time point is the ganglion tf the vth cranial nerve.

FIG. 7

Expression of Medane and TH in coronal sections of E14.5 embryos. At E14.5 Medane is still strongly expressed in the ventricular zone of the striatum (str), discrete points in the ventricular zone of the thalamus (th) and the hypothalamus (hyp), the zone inserta (zi) and the dorsal part of the mesencephalic vesicle, representing the superior and inferior colliculus (SC, IC, 1-12). Compared to the TH-expression it is not expressed in the olfactory bulb (ob) and not any more in the substantia nigra, the locus coerulus, and caudal noradrenergic groups (na, 1″-12″). This expression pattern is consistent with the idea, that Medane expression precedes TH-expression in cells destined to become catecholaminergic cells.

FIG. 8

Expression of Medane in a mid sagital section of an E16.5 embryo. Expression of Medane is now restricted to the ventricular and subventricular zone of the striatum (str), a region where the neurons migrating to the olfactory bulb (ob) are located. Indeed, Medane expressing cells can be found along the so called rostral migratory stream just until there entrance into the olfactory bulb. Again this fits the idea, that cells destined to become TH-positive cells express Medane before they express TH (cx=cortex, hc=hippocampus).

FIG. 9

Expression of Medane in coronal sections of an E18.5 embryo.

Like at E16.5 expression of Medane is now restricted to the ventricular and subventricular zone of the striatum (str), that is the rostral migratory stream but can now also be found in the lower layers of the developing somatosensory cortex (str=striatum, cx=cortex, III=third ventricle). The significance of the expression of Medane in the lower layers of the somatosensory cortex, which persists into adulthood remains unclear.

FIG. 10

Expression of Medane in coronal sections of and adult mouse. Expression of Medane can now only be found in dispersed cells in the lower layers of the somatosensory cortex (not shown) and in single cells (arrows) around the third ventricle (3^{rd}) and the olfactory ventricle (ov), the reminiscent of the rostral migratory stream of embryonic development and the location of neuronal stem cells. Specifically this location supports the idea, that cells expressing Medane are the progenitors of the TH-expressing interneurons of the olfactory bulb, which are generated persistently during adulthood.

FIG. 11

Ectopic specification of TH-expressing cells by Mdn in Zebrafish embryos. a) control embryos injected only with GFP RNA showing TH-expressing cells in the lateral midline of the diencephalon. b+d) Lateral views (b=right and d=left) of injected embryos with Mdn RNA showing a 10 fold presence of ectopic TH-expressing cells in the lateral midline of Diencephalon. c) A new cluster of cells with neuronal morphology expressing TH are shown in the ventral midline of the diencephalon in an injected embryos with Mdn RNA.

FIG. 12

Expression of Medane and TH in the neuroepithelium. Expression of Mdn and TH in the neuroepithelium at E12. Note that Mdn expression can be found in cells close to the ventricular surface, in the ventricular zone (VZ), whereas TH expressing cells are found more distal to the ventricular zone in the differentiating zone (DZ) of the neuroepithelium. The expression domains overlap in the differentiating zone. This spatial distribution of the two expression domains fits the hypothesis that Mdn is expressed in dopaminergic neuronal precursor cells which then migrate out of the ventricular zone while differentiating thereby taking on the dopaminergic-TH-expressing phenotype.

### DETAILED DESCRIPTION OF THE INVENTION

Genomic Structure of Mdn

As a result of the searching for new transcription factors specifying dopaminergic neurons, we have performed RT-PCR with degenerate primers from the conserved bHLH domain of hairy/E (spl) related genes. Sequencing results shows the PCR-generated insert of H2 clone was derived from the mRNA of a new bHLH protein. Database searches with the deduced amino acid sequence revealed that the predicted bHLH region of clone H2 is a novel protein and related to proteins of the Drosophila hairy and E (spl) family.

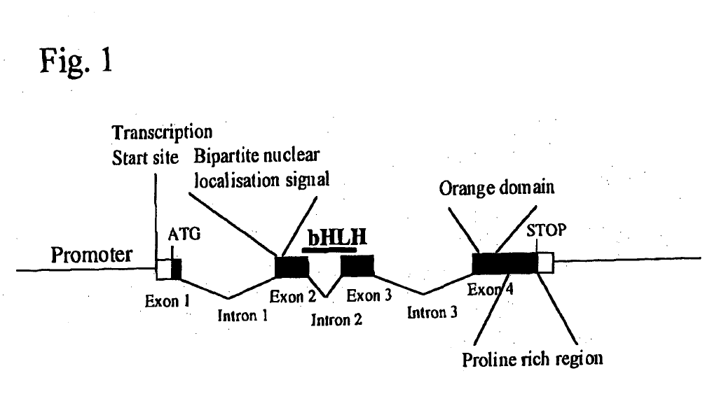

The Mdn transcript is distributed over 4 exons and the gene spans 1000 kb of genomic DNA. The genomic organisation of Mdn is shown in FIG. 1. All the introns were located within the coding region. Analysis of the DNA sequences at the intron-exon boundaries of Mdn showed that they all adhere to the 5′gt/ag3′ splice junction consensus rule for donor and acceptor splice sites (Breathnach and Chambon, 1981; Shapiro and Senapathy, 1987). Southern blot analysis of total mouse genomic DNA digested with HindIII showed a single band when hybridised with the full-length cDNA of Mdn, under non-stringent conditions. Therefore, Mdn is a single-copy gene per haploid.

The exons of MDN range in size from 97 to 687 bp and the size of the introns vary from 220 to 453 bp. Primes were designed from intronic sequences to allow amplification of each exon. The amplification products were designed to be fewer that 400 bp in length in order to facilitate their use in SSCA/heteroduplex protocols for mutational analyses. The primer sequences, product sizes, and annealing conditions are shown in Table 1.

A single start site was detected for Mdn when RACE and cDNA library screening were performed as described in the Examples. We designated this nucleotide the start (+1) of the transcript. We were never able to obtain a product when primers MdnPr1D or MdnPr2D (20-100 bp upstream of the transcription start site, respectively) were used in combination with primer H2.10R (exon 2) on RNA template. Therefore, it seems unlikely that there is any RNA species containing these sequences 5′ of our designated first exon.

Computer analysis of the region usptream of the transcription initiation site of Mdn transcript predicts a TATA box (position −36). 1500 bp sequence between the 5′ proximal region of transcription start site and part of intron 1 of Mdn there is a GC-rich region containing three stretches of DNA that satisfy the criteria for CpG islands were found (a ratio of observed/expected CpG>0.6 and a content of G+C>50. A search of the 5′-flanking regions of Mdn for cis-acting regulatory elements revealed three putative Spl-binding sites. There were also four recognitions sites for N-boxes, six sites for E-boxes (class A, B and C) and one for RBP-JK (direct target of Notch). Concerning the 3′ UTR of Mdn, two canonical polyadenylation signal sequences AATAAA were present 21 and 46 bp upstream of the polyadenylation site.

Primary and Secondary Amino Acid Structure of Mdn/MDN

Mdn/MDN cDNAs contain a 723 bp open reading frame starting from the first ATG codon present at nucleotide residue 85. Since it is the only ATG codon upstream the bHLH, the first Methionine is assigned as the initiation codon.

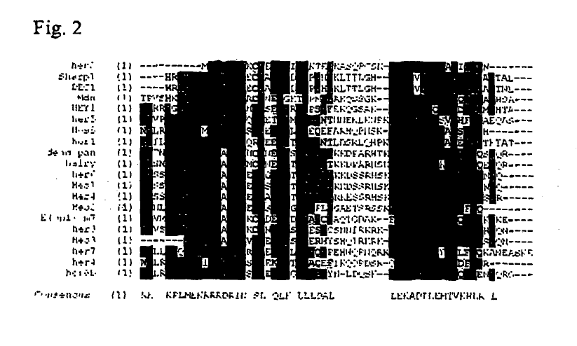

Mdn encoded a proteins of 241 amino acids residues in length, and the calculated molecular mass is estimated to be 27.042 kD. The bHLH of Mdn (amino acid residues 11-59) shows 57/63% similarity and 49/43% identity to the hairy/E (spl) gene products, respectively (FIG. 2).

Two additional helices (III and IV, referred to as “orange domain”) present in hairy and E (spl), were also found in the corresponding position of Mdn. Comparison analysis between Mdn and MDN proteins shows a similarity of 93.4% and an identity of 91.4%. The bipartite nuclear translocation signals, the bHLH domain, the orange domain, and the proline rich region as well (20% residues encoded by the portion between amino acid residues 132-242) are conserved between human and mouse Medane genes. During sequencing, we identified an amino acid polymorphism in the coding region of Mdn: aa Pro156 (CCG or CCA). Another polymorphisms were detected in the 3′UTR: nt 838 (C or T). These changes may be useful as allelespecific polymorphisms for use in linkage desequilibrium studies.

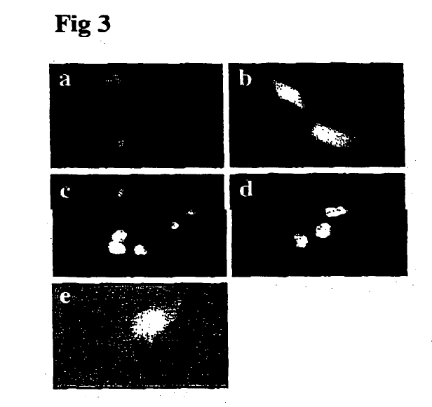

Since Mdn protein harbours a putative bipartite nuclear translocation signal in its N-terminal region, we further attempted to evaluate the functional significance of this putative domain. A human cell line were transient transfected with a GFP-tagged Mdn-coding vector. Control transfections with the blank vector (GFP) resulted. The signal was observed throughout the cell while cells transfected with Mdn-GFP recombinant protein showed a fluorescence signal only in the nucleus (FIG. 3).

Mapping Data

Results of mapping with the T31 Radiation Hybrid (RH) panel localized Mdn 15.9 cR from marker D8Mit297 on mouse chromosome 8, between markers D8Mit297 and D8Mit98.

Concerning the human gene, framework mapping of MDN was first established by G3 mapping panel. This studies places MDN on human chromosome 4 between markers WI-4886 and AFMA239XA5. Given the importance of genes mapping to telomeric positions, to achieve an even more precise localization, we performed a G3 RH mapping panel. This last panel mapped also MDN in the telomeric region of human chromosome 4, between markers (SHGC4-3 and SHGC-63497). Moreover, both mapping data for Mdn and MDN loci in mouse and human chromosomes, respectively, agrees with the syntenic region established for these two chromosomal positions.

Expression Pattern of Mdn

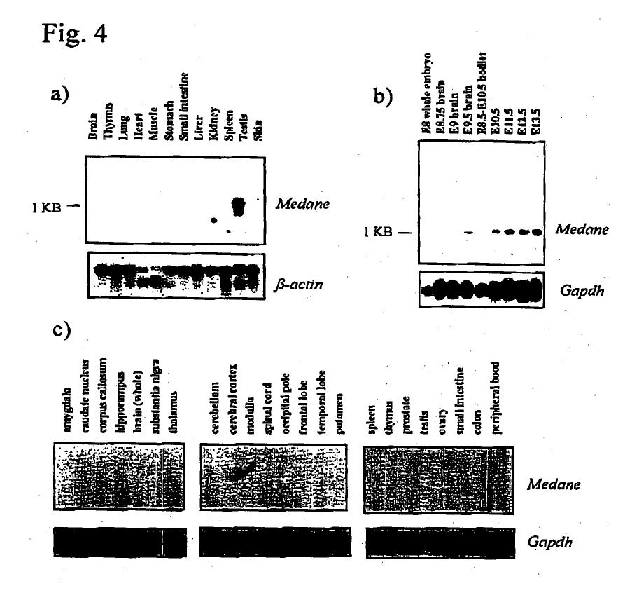

In order to analyse the expression pattern of Mdn in the developing embryo and in the adult we performed in situ hybridisation on whole embryos (E9.0-E11.5) and on sections (E12.5-adult). Expression of Mdn is nearly exclusively restricted to the CNS. The only other expression domains are the vomeronasal organ, dispersed cells in the olfactory epithelium and in adult testis (FIG. 4).

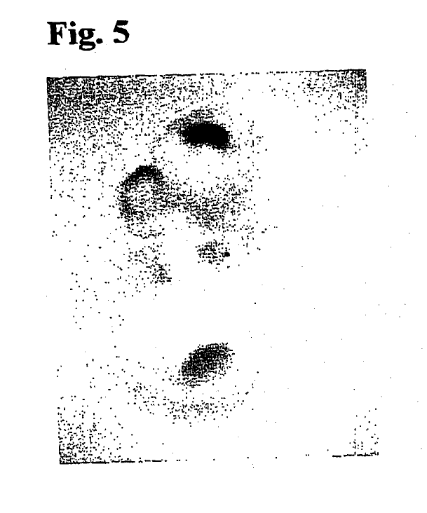

To determine whether Mdn is indeed expressed in the dopaminergic neuronal precursors, its expression was analysed in various mouse tissues, then studied the correlation with the expression of TH (a hallmark protein of catecholaminergic system). Transcription of Mdn is first detected at low levels at E9. At E9-10.5 mouse stages the transcript is restricted to the developing ventral mesencephalon anterior to the isthmic organiser (FIG. 5). This is the region where 2.5 days later dopaminergic neurons will develop, as determined by the presence of TH+cells.

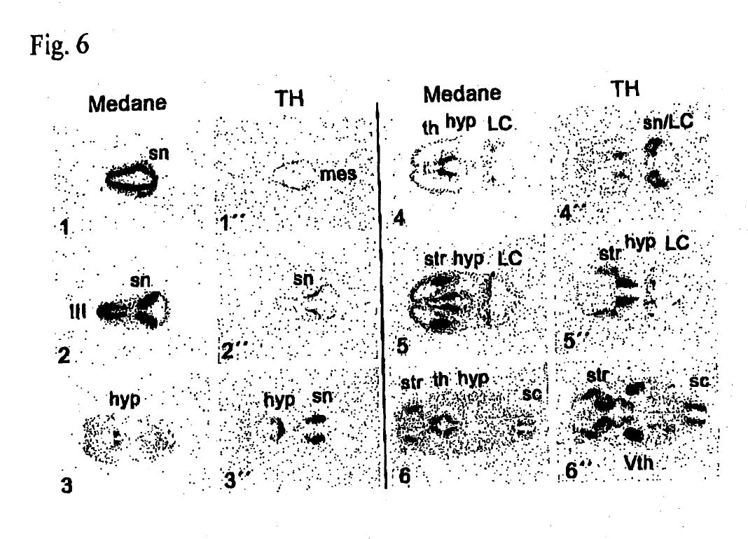

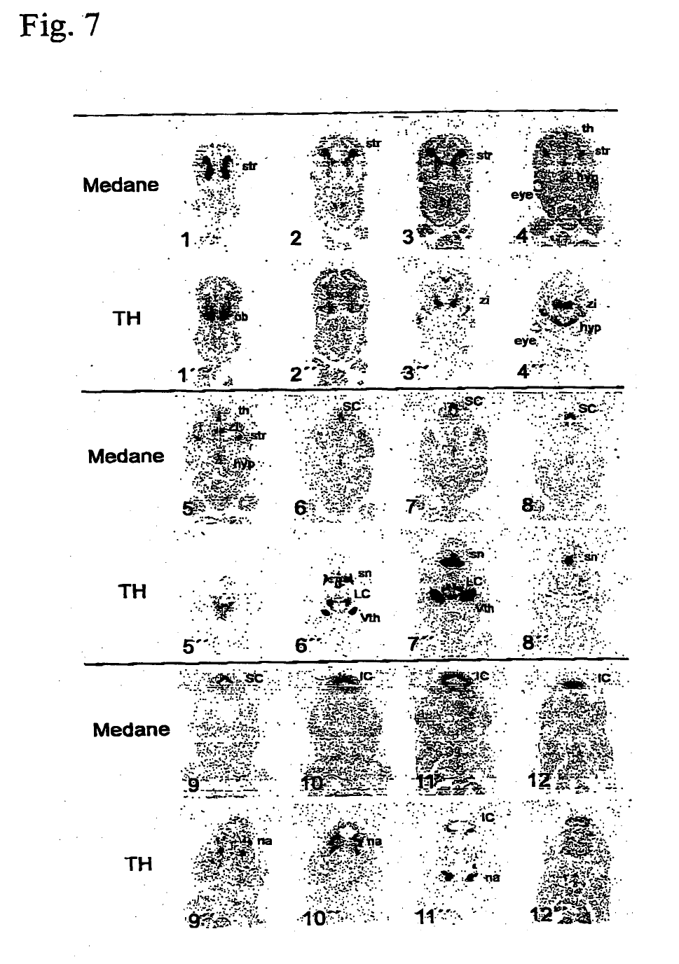

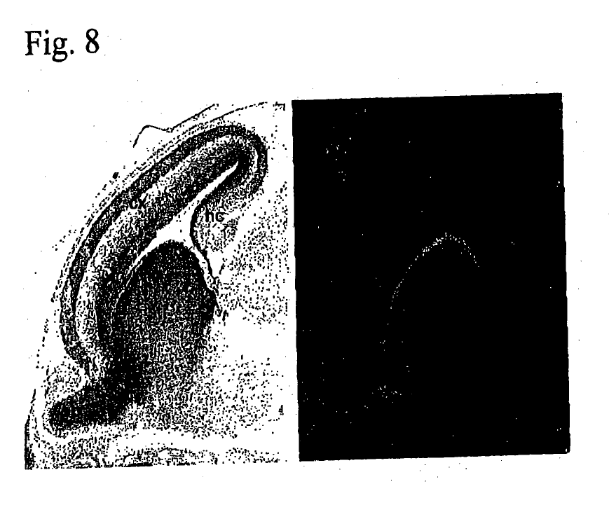

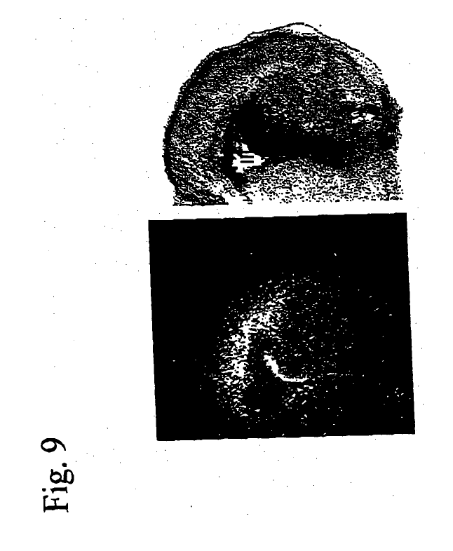

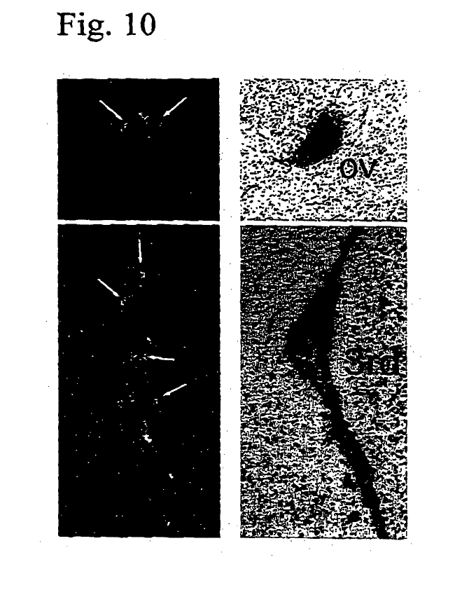

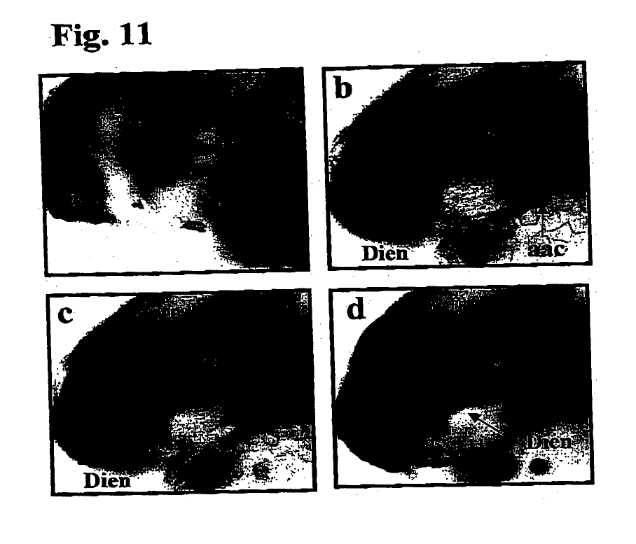

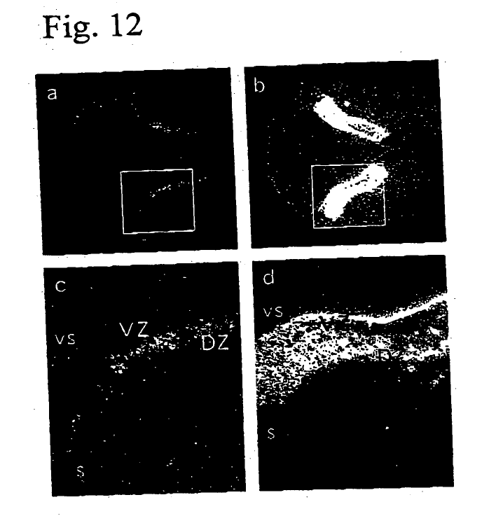

During the subsequent development of the CNS further Mdn expression domains can be found. At E12 (FIG. 6) Mdn is still highly expressed in the ventral mesencephalon, the future substantia nigra (sn). However, now it can also be found in the dorsal part of the 3^{rd} ventricle, the hypothalamus, the developing striatum and in dorsal root ganglia. All these regions are characterized by the onset of tyrosine hydroxylase expression around this time (FIG. 6). Interestingly expression of Mdn starts to cease in these domains once TH-expression becomes prominent. E.g. at E14.5 (FIG. 7) TH-expression is strong in the substantia nigra (sn) but now Mdn expression is absent in this region. During further development Mdn also ceased to be expressed in all other expression domains except in the subventricular zone (SVZ) (FIGS. 8, 9), a germinal region which continuously generates new neurons destined to the olfactory bulb even during adulthood (Temple and Alvarez-Buylla, 1999). Neurons from the subventricular zone migrate along the rostral migratory stream (RMS), then differentiate into local interneurons and finally reach the olfactory bulb (Luskin, 1993, Lois and alvarez-Buylla, 1994; Doetsch and Alvarez-buylla, 1996), where a large part of them differentiate into TH-expressing neurons. At E16 and E18 Mdn expression is very prominent in the RMS, however, not in the olfactory bulb. In adult brain, Mdn can still be found in dispersed cells in the lower layers of the somatosensory cortex and in single cells around the 3^{rd} ventricle and the olfactory ventricle (FIG. 10) representing the location of neuronal stem cells and the RMS. Thus, during development and in adulthood Mdn is expressed only in regions where subsequently TH positive dopaminergic neurons will arise.

Ectopic Expression of Mdn in Zebrafish

The correlation between Mdn and TH expression suggested that Mdn could specify DA neurons. To test this hypothesis, we expressed Mdn ectopically in the zebrafish. After injection of capped Mdn RNA into 16-cell zebrafish embryos, we found a 2-10 fold increase in the number of TH-expressing cells in the normally TH positive diencephalon cluster (100% of cases, n=20), but also new cluster of cells with neuronal morphology that express TH were found in the ventral midline of the diencephalon (FIG. 10). To discard the possibility that injection of Mdn induce general neurogenesis by mimicking the function of other bHLH rather than specific induction of DA neurons, we tested for ngnl expression by in situ hybridisation. No general induction of neurogenesis was detected in any embryo.

The following examples are set forth for illustrative purposes and should not be considered as limiting the scope of protection of the present invention.

### EXAMPLES

Cloning of Mdn Gene Transcript.

A clone containing a partial Mdn cDNA resulted from the application of RT-PCR approach using degenerated primers.

The mouse foetal cDNA source was prepared as follows: ventral part of the midbrain from mouse embryonic day 8-13.5 (E8-13.5) was mixed prior to RNA isolation. In parallel experiments, RNA from the rest of the brain and body was isolated as well. Total RNA was extracted using RNeasy Mini Kit from Qiagen (according the manufacturer's recommendations) followed by poly(A)⁺ RNA selection, using Dynabeads Oligo (dT)₂₅ (Dynabeads mRNA purification kit).

Furthermore, first-strand synthesis is carried out by Moloney Murine Leukemia Virus (M-MuLV) reverse transcriptase (Amersham Pharmacia). After an annealing step of 5 minutes at room temperature of the poly(A)⁺ RNA from the mentioned tissues with an oligo pd (N)₆, the reaction is performed at 37° C. for 1 hr. This single strand cDNA was used as a template to carry out an RT-PCR with degenerated primers. The primers were designed on the basis of the conserved amino acids from the bHLH domain of Drosophila hairy and its related protein her5 of zebrafish (Müller et al., 1996). Consequently, fully degenerate primers directed to conserved amino acids from the basic-helix-I of hairy (RRRARIN) and from her5 (RRRDRIN) proteins, as well as reverse primers from helix-II of hairy (EKADILE) and from her5 (EKAEILE) were designed. The third codon positions of fourfold degeneracy were substituted by inosine. Combinations of forward against reverse degenerated primers were subjected to PCR in different experiments. Briefly, cDNA from the ventral part of the midbrain and from the rest of the brain and body as well of a mouse embryo E9-10 were used as a template in parallel experiments. Conditions for the hot-start PCR were 94° C. for 4 min, then 95° C. for 30 s, 52° C. for 1 min, 72° C. for 50 s for 32 cycles followed by a 5-min extension at 74° C. in 50 μl. PCR products were purified through RCR-column (Qiagen) and then one-fiftieth of the first PCR products were subjected to a second round of amplification of 33 cycles at 60° C. of annealing temperature. In this second PCR, an EcoRI and a XbaI site was added to the 5′ forward and reverse degenerate primers, respectively, for further subcloning in pBluescript vector. PCR products were electrophoresed on a 1.5% agarose gel. The cDNAs amplified from the ventral part of the midbrain were compared with the cDNAs obtained from the rest of the E9-10 embryo. Those that appeared to be specific and unique for the ventral part of the midbrain were subsequently double cut with EcoRI and XbaI restriction enzymes, then purified by phenol-chloroform extraction and finally subcloned into EcoRI-XbaI of pBluescript vector. Colonies were gridded in duplicate onto nylon filters. To detect clones containing a bHLH related to hairy or her5 proteins, the filter was hybridizised with an oligomer covering the bHLH domain of hairy or her5, respectively. A gradient of stringency washes in distinct experiments was done to detect those clones with higher similarity to the hairy or her5 bHLH domain. Positives clones were sequenced with M13D and M13R primers using fluorescent DyeDeoxy Terminators on an ABI373A automatic DNA sequencer (Applied Biosystem).

As a consequence of the RT-PCR approach, we detected a positive recombinant clone (clone H2). This new clone contained a novel EST, which turned out to be a partial cDNA of a new gene encoding a bHLH we call Medane (Mdn). The sequence data of the mouse H2 clone are as follows: 5′agaaggagagaccgaattaaccgctgcttgaacgagctgggcaagacagtccct atggccctggcgaaacagagttccgggaaactggagaaggcggagatcctggag3′

Full-length cDNA of Mdn Gene

The partial cDNA obtained from clone H2 was then used to obtain the full-length of Mdn transcript in parallel approaches: a) Rapid amplifications of cDNA ends (RACE). 5′ RACE was carried out using the Marathon-Ready cDNA Amplification Kit (Clontech) from mouse 11-day embryo, with the Mdn-specific primers H2-9R and nested primer H2-8R. The products were subcloned into pBluescript TVector for sequencing. T-vector was prepared essentially as described by Marchuk et al., (1991). 3′ RACE was performed according to Frohman, (1993) using primer QT₂₀ in the first-strand cDNA synthesis reaction from mouse 9-10-day embryo poly(A)⁺ RNA. The cDNA products were submitted to cycle amplification. PCR was carried out using Q₀ primer and the Mdn-specific primer H2.1. A second round of PCR was performed with Q₁ and nested specific primer H2.3. Products were subcloned in pBluescript TVector and sequenced. Clone 200A reveals the 5′ UTR whereas clone H2-A4 reveals the 3′ UTR of Mdn; b) cDNA library screening: Approximately 4×10⁶ recombinant phages from a mouse 11-day embryo cDNA library in lambda TriplEx vector (Clontech), were screened with 873 bp PCR product derived from clone H2-A4 containing the 3′ part of the coding region and the 3′UTR of Mdn as well (base pairs 166-1000). Hybridisation was in 0.5 M sodium phosphate, pH 7.2/7% SDS, at 65° C. overnight. Membranes were washed with 2× SSC/0.5% SDS for 15 min at 45° C., 1× SSC/0.5 SDS for 30 min at 65° C., and 0.2× SSC/0.5% SDS for 30 min at 65° C. Three isolated phages revealed to contain the putative full length of Mdn transcript.

To identify the human homologue of Mdn gene, approximately 8×10⁶ recombinant phages from a human foetal brain CDNA library in lambda gt10 (Clontech) were screened with the 873 bp PCR product described above.

The conditions of hybridisation and washing were also as describe above. Two positive clones containing the cDNA of MDN were found by sequencing.

Database Search

The recent publication of the entire heterochromatic sequence of Drosophila melanogaster and C. elegans, allowed us to look for the counterpart of Mdn gene in this organisms. We used the BLAST search (Altschul et al., 1997) programs available on http://www.ncbi.nlm.nih.gov to look for the ortologue of Mdn in Drosophila and in C.elegans databases. The nucleotide and amino acid sequences corresponding to the bHLH domain of Mdn were used as a query.

Exon Identification and Amplification

BAC genomic clones containing Mdn/MDN loci were isolated by screening of a Mouse/Human BAC genomic library (Resource Center of the German Human Genome Project-DHGP) when a partial cDNA of Mdn/MDN (base pairs 166-1000) was used as a probe, respectively. BAC-DNA preparation from the positive BACs were obtained and prepared for direct sequencing. Intron-Exon boundaries were identified and the precise lengths of the introns of the mouse and human Medane gene were determined by comparing the Mdn/MDN cDNA sequences to the genomic DNA sequences obtained from a mouse/human Medane-containing BAC, respectively. To check the intron/exon structure of Mdn/MDN, we designed a set of primers covering the entire cDNA. Combinations of primers that have identical sized bands using the cDNA and genomic DNA as templates indicated a lack of an intervening intron between the two primers. The presence of an intron was indicated by a discrepancy in the size of PCR product produced by using genomic and cDNA templates. Those PCR products with primer combinations that indicated the presence of an intron were then used for sequencing in order to identify the splice donor and acceptor sites. In order to allow mutational screening of MDN gene, intronic primers were designed to amplify each exon (table 1), tested on human genomic DNA and the products sequenced with nested primers. PCR was performed in 50 μl volumes with 200 ng of human genomic DNA, 10 pmols of each primer, 1.25 μM dNTPs, 1.5 mM MgCl₂, 1 U of Taq polymerase (Fermentas) and 6% DMSO. PCR amplifications were performed in an eppendorf thermal cycler, using a hot-start procedure. Initial denaturation of samples was at 95° C. for 4 min followed by 33 cycles at 56° C. annealing temperature for all PCR primer pairs.

Whole Mount in Situ Hybridisation

Pregnant mice were killed by cervical dislocation, embryos were dissected, fixed overnight at 4° C. in 4% paraformaldehyde. Fixed embryos and brains from different stages (E8.5-E18) were treated and whole mount in situ hybridisation were performed as described (Sporle et al., 1998). Antisense and sense digoxigening (DIG)-labelled riboprobes for Mdn (base pairs 166-1000) were produced using a DIG-RNA labelling kit (Boehringer-Mannheim), following the manufacturer's instructions.

Histological Analysis

Brains of embryonic mice or whole embryos were either transcardially perfused or immersion fixed overnight at 4° C. in 4% paraformaldehyde. Some of the adult brains were shock frozen on dry ice. Perfused brains were either cut on a cryostat in 30 μm thick sections or paraffin embedded and cut on a microtome in 4-8 μm thick sections. Frozen brains were cut on a cryostat in 18 μm thick sections and processed for in situ hybridisation. in situ hybridisation of frozen and paraffin sections was performed after a modified method of Dagerlind et al. (1993). Antisense and sense mRNA probes transcribed from linearized plasmids containing fragments of TH (base pairs 23-788), and Mdn (base pairs 166-1000) were used as a probe. Following in situ hybridisation, sections were counterstained with cresylviolet according to a modified method by Nissl (1894).

Northern Analysis

Poly(A)⁺ RNA from mouse 8.75-15-days embryos was prepared as described above. 2.5 μg of poly(A)⁺ RNA and 3 μg of RNA ladder (0.24-9.5 kb RNA Ladder; GibcoBRL) were electrophoresed on a 1.2% agarose/formaldehyde gel and then transferred onto a nylon membrane (Hybond-N⁺, Amersham Pharmacia) in 20× SSC. The filters were hybridised overnight at 65° C. in 0.5 M sodium phosphate buffer, pH 7, 2/7% SDS with a partial Mdn cDNA probe (base pairs 166-1000). To check equal loading of RNAs, Northern were reprobed with the GAPDH CDNA. The membrane was washed with 2×SSC/0.5% SDS for 15 min at 45°C, 1× SSC/0.5 SDS for 30 min at 65° C., and 0.5× SSC/0.5% SDS for 20 min at 65° C. and exposed to X-ray film with intensifiers for 7 days. The mouse (Origene) and human (Clontech) adult multipletissue Northern blots containing 2 μg of poly(A)⁺ RNA from the tissues indicated were hybridised with a probe containing a partial cDNA fragment of Mdn/MDN cDNA, respectively (base pairs 166-1000). Hybridisation was performed at 65° C. for 1.5 hr in ExpressHyb hybridisation solution (Clontech), according to the protocol provided. The membranes were washed twice in 3× SSC/0.1% SDS at 37° C. for 20 min, and then in 2× SSC/0.1% SDS at 50° C. for 20 min and 1× SSC/0.1% SDS at 65° C. for 10 min, and exposed to an X-ray film with intensifiers for 5 days. To check equal loading of RNAs, all Northern blots were reprobed with a GAPDH cDNA, with an exception for the mouse adult Northern blot which was reprobed with a β-actin cDNA.

Southern Blot

Mouse genomic DNA was digested with Hind III and electrophoresed on 0.8% agarose gel, then blotted onto nylon membrane (Hybond-N⁺, Amersham Pharmacia). Finally hybridizised with the entire cDNA of Mdn. Conditions for hybridisation and washing were identical to described above.

Radiation Hybrid Mapping Panel.

The radiation hybrid (RH) 100 cell lines DNAs of the T31 mouse/hamster panel (Jackson laboratory) were tested plus parental controls. A 212 bp fragment was amplified by PCR using forward primer H2.18D and reverse primer H2.19R from the first intron of Mdn to enhance specificity. PCR was performed under standard conditions in 50 μl volumes with five μl of RH DNAs. The PCR cycling profile was: 94° C. 3 min, (94° C. 30 s, 55° C. 35 s, 72° C. 30 s) 40 times, 72° C. 7 min, 4° C. hold. In all cases, the hamster background gave rise no product, making scoring unambiguous.

Since radiation hybrid mapping is a +/− PCR assay and false positive and false negative reactions may distort the linkage, each cell line was typed twice. Any line that give a new (−) in a string of previously linked (+), or vice versa, were retyped to determine the final correct score for each cell line. Hybrids that gave a signal in both PCR reactions were scored as positive and those giving no signal in both as negative. PCR products were electrophoresed using very sensitive detection conditions on agarose gel and the data were analysed as positive, negative, but not missing, since all the lines were checked using GAPDH primers. Results were submitted to the Whitehead mouse RH map website for automated mapping data analysis.

Concerning the mapping of the human gene, the Stanford G3 mapping panel of 83 RH clones of the whole human genome was used to map the MDN locus. Primers used were H5D and H4R. Conditions and analysis were carried out as described above. A server for the chromosome localization of MDN was used at http://www-shgc.stanford.edu

The Genebridge 4 mapping panel of 93 RH clones of the whole human genome was also performed, using the conditions described above. Chromosome localisation of Mdn was performed by accessing the server available at http://www-genome.wi.mit.edu/cgi-bin/contig/rhmapper.pl

Subcellular Localization of Mdn

A Mdn-GFP fusion protein was created by subcloning the coding region of Mdn into pEGFP-N1 vector (Clontech) so that it is in frame with the EGFP coding sequences, with no intervening in-frame stop codons, allowing the localisation of the fusion protein in vivo. Human embryonic kidney 293 cell line (Graham et al., 1977; ATTC-Nr.CRL-1573) were cultured in Dulbecco's modified Eagle medium (DMEM) with 10% foetal calf serum and 1% Pen/Strep. 30-40% confluence plates of exponential growing cells were transfected with 0.5 μg Mdn-GFP fusion protein using standard transfections methods with 2.5 M of calcium phosphate (Graham F L and Eb A J van der, 1973). Control transfections were carried out with 0.5 μg of blank vector (pEGFP-N1). The following day, the subcellular localization of the recombinant fusion protein and control was visualized by direct fluorescence of intact cells.

Injection of Mdn in Zebrafish Embryos

For this, 16 cell-stage zebrafish embryos were injected with Mdn capped mRNA and the presence of ectopic TH-expressing cells was determined 16 hr later. Capped mRNAs of Mdn were synthesised (Ambion), then verified by in vitro translation (Ambion) and the translation product was checked in a SDS-PAGE gel. Embryos were obtained from natural spawning of wild-type adults. Injections were carried out into one central blastomere of the 16-celled embryo (50 pg), together with the lineage tracer GFP MRNA (50 pg). 12 hrs after injection, the presence of Mdn RNA was checked by in situ hybridisation of fixed embryos using a antisense cDNA of Mdn as a probe (base pairs 166-1000). Only embryos having received the injection in the CNS (sorted out at 16 hrs under fluorescence) were analysed. Phenotypic analyses, in situ hybridisation and immunocytochemistry were done following standard protocols (Hauptmann and Gerster, 1994). For the immunodetection of TH, a polyclonal anti-TH antibody (Chemicon) was used at 1/1000. For detection of neurogenin1 (ngn1) RNA, a ngn1probe was used (Blader et al., 1997).

Knock-out Targeting Strategy (Animal Model)

Firstly, a targeting vector containing a mutated allele of Medane is designed to recombine specifically with the Medane locus. The components of such a vector are sequences which are homologous with the desired chromosomal integration site of Medane. For the generation of the 5′ arm, a fragment of isogenic homologous sequences of 1758 bp was used, including also the first exon and the first intron. The 3′ arm was a SmaI-Eco47III fragment (5245 bp) containing the 3^{rd} exon and the entire 3′ UTR of Mdn. Other components were also included in the vector such as the beta-galactosidase gene (lacZ) as a reporter gene for the Medane expression, PGK neo gene (neomycin phosphotransferase) as a positive marker, and the PGK tk gene (thymidine kinase) as a negative selector markers. These markers provide strong selection for the clones that have been targeted in the right locus by homologous recombination event. The neo marker was surrounded with lopxp sites to allow site-specific recombination in mice. This technique makes possible to generate a germline mutation without the positive selection marker using Cre-loxP system. The vector is linearized outside the homologous sequences before transfection into ES cells. To confirm that the desired genetic change has occurred, a diagnostic Southern screening strategy was designed. Therefore, to analyse both the 5′ and 3′ aspects of the target locus, we used 5′ and 3′ external probes from sequences flanking both ends of the homologous sequences. Pluripotent embryonic stem (ES) cells derived from a mixed 129SV background were electroporated with the targeting vector for introducing the mutated Medane allele into ES cells, then plate them under double selection (Gancyclovir and G418) in feeder plates. Homologous recombination events were detected by genotyping using BamHI digestion and southern blot analysis. The final recombinant allele (Medane is mutated) raised from the desired genetic exchange as a consequence of double reciprocal recombination event which takes place between the vector and the chromosomal sequences. In this way, the wild type Medane allele is replaced by all the components of the vector which are between the 5′ and 3′ homologous sequences. The heterologous sequences at the ends of this arms of homology are excised following targeting. In this way, a mutant cell is created lacking the sequence of the gene encoding for the nuclear translocation signal, the bHLH domain of Medane, and the second intron as well.

Blastocyst recovered from pregnant superovulated females were injected with the Medane-mutant ES cells and transferred into a pseudopregnant host female. Germ-line transmission is now determined by PCR and Southern blot analysis of tail DNA. Chimeras were bred with C57BL mice to obtain F₁ offspring. Heterozygotes (Mdn^{+/-}) for the targeted allele can be mated together to produce F₂ litters with wild-type (Mdn^{+/+}), heterozygote (Mdn^{+/-}), and homozygotes (Mdn^{-/-}) for analysis.

Primers and Probes

| | |
|---|---|
| H2.1: | (5′-tcgctgcttgaacgagctg-3′); |
| H2.10R: | 5′-cagagttccgggaaactg-3′; |
| H2 18D: | (5′-gagactggaaggagagtcc-3′); |
| H2.19R: | (5′-agggtcactaattcgccaac-3′); |
| H2.3: | (5′-tggcaagacagtccctatgg-3′); |
| H4R: | (5′-ctggttccacctccttctc-3′); |
| H5D: | (5′-ccgctagaagttctgctgg-3′); |
| MdnPr1D: | (5′-ggagccccctcggacct-3′); |
| MdnPr2D: | (5′-caaacgcagaactcctaatcc-3′); |
| MdnPr1D: | (5′-ggagccccctcggacct-3′); |
| MdnPr2D: | (5′-caaacgcagaactcctaatcc-3′) |

It is still a major challenge to understand how dopaminergic neurons are specified and assigned their fate in the vertebrate CNS. In a search for bHLH-containing transcription factors that might function as intracellular mediators for the specification of dopaminergic neuronal lineage in the vertebrate CNS, we have isolated, characterised and mapped a new murine gene and its human counterpart. The cDNA of the new gene, we termed Medane (Mdn) (for Mesencephalic Dopaminergic neurons E (spl) and hairy related gene), encodes a bHLH protein related to the products of hairy and E (spl) genes of Drosophila.

Most of the genes governing the choice of neural fate from multipotent progenitors cells in a variety of tissues and organisms (reviewed by Garrell and Campuzano, 1991) are bHLH proteins. There is evidence that mammalian homologues of Drosophila bHLH play and important role as a regulators of cell fate decisions in the developing nervous system and inducers of neuronal differentiation at the level of gene transcription (Reviewed by Jan and Jan, 1993; Lee, 1997).

Mdn protein share high structural similarities in the bHLH region with several cDNAs encoding proteins of the hairy-E (slp) family (FIG. 2) that have been cloned in mice, rat and human, including HES (Akazaka et al., 1992; Sasai et al., 1992), SHARP (Rossner et al., 1997), HRT (Nakagawa et al., 1999), DEC1 (Shen et al., 1997) subclasses, but have characteristics that are distinct from those mentioned above. Mdn differs from Hairy/E (spl) and HES transcription factors by the absence of both the proline residue in its basic DNAbinding domain, and the carboxy-terminal WRPW amino acid motif. In both Drosophila and vertebrates, these features have been proposed to confer unconventional DNAbinding specificity to bHLH proteins and to permit the recruitment of Groucho-like cotranscriptional repressors, respectively (Fischer and Caudy, 1998, and references therein). We specifically note Mdn is divergent in several critical and conserved amino acid positions in the bHLH domain characteristic within the SHARP, HRT, HEY and DEC subclass. In addition, the full-length transcript of Mdn shows that the similarity does not extents into the N-terminus and C-terminus of other hairy/E (spl)-related genes. This observation argues in favour that Mdn constitute a new subclass of bHLH transcription factor distinct and closely related gene of hairy and E (spl). Then, we termed the gene as Medane to emphasise the distant features of this new gene and the previously cloned mammalian hairy-E (slp) related proteins.

Expression of Mdn mRNA is detected in a very dynamic pattern in the embryonic CNS. To analyse the tissue distribution and the ontogenetic expression pattern, we performed whole mount in situ and cryosections experiments. Transcription is first detected at low level and during early embryonic mouse 9-day (E9) strikingly restricted to the proliferative neuroepithelium of the developing ventral mesencephalon, where dopaminergic neurons develop. This early starting appearance of Mdn in the ventral part of the midbrain coincides with the observation that the progenitors for mesDA neurons also lie on this part of the mouse embryo as early as E9 (Hynes and Rosenthal, 1999). The other intracellular molecules that are known to be implicated in the mesDA pathway, Ptx3 and Nurrl, start to be expressed in the mouse mesDA territory at E10.5/E11, respectively. In contrast, Mdn is uniquely expressed in the mesDA system as early as E9, when the progenitors for this neuronal cell type start to differentiate into DA (Hynes and Rosenthal, 1999) and became TH⁺ cells at E11.5.

Interestingly, in contrast to Nurr1 and Pxt3 genes, which none of them are capable to induce dopaminergic fate in embryonic explants (Hynes and Rosenthal, 1999), Mdn is capable to specify DA neurons when expressed ectopically, may be activating a program for DA-specific gene expression and differentiation. These data suggest that Ptx3 and Nurr1 for a regulatory cascade for development of the mesDA system in which Mdn may act as an upstream activator.

As determined by in situ hybridisation with TH cRNA probe, Mdn mRNA expression was detected in a spatially correlated distribution, although TH appears slightly later when differentiating cells are presumed to have migrated further away. Detailed analysis of in situ hybridisation experiments on consecutive sections either incubated with the ³⁵S-Medane probe or the ³⁵S-TH-probe also revealed a spatial correlation between Mdn and TH expressing cells. Whereas Mdn is expressed predominantly close to the ventricular surface where neuronal progenitors are located, TH expression can predominantly be found in cell layers more distal to the ventricular surface, the differentiating zone (DZ). A layer of overlap between the two expression domains can be found (FIG. 12). This supports the idea that Mdn is expressed in dopaminergic precursor cells but once these cells start to differentiate further they migrate away from the ventricular surface and lose Mdn expression. The expression of Mdn in the RMS but not in the olfactory bulb during late embryonic development and around the 3^{rd} ventricle in adulthood also fits this idea: since once the cells have entered the bulb they enter a more differentiated state, e.g. start to express TH.

Taken together, the temporal-spatial expression pattern of Mdn strongly suggests that it is expressed in dopaminergic precursor cells during development as well as in adult mice. Moreover, given the close association between Mdn and TH expression in developing DA system and the observation that Mdn expression does not overlap TH expression in the adult mouse CNS, it is likely that Mdn is involved in the specification but not in the maintenance of this subset of dopaminergic neurons. Taken together our results about the expression of Mdn, it is likely that Mdn is rather involved in the early events of development of the mammalian DA system, but Nurrl and Pxt3 in the late phases. Early steps include the generation of the appropriate numbers of neuronal and glial precursors and the migration of precommitted cells to their final position while late events encompass axonal outgrowth, dendritic arborisation, synaptogenesis.

Given the fact that expression of Mdn in zefrafish triggered the aparition of ectopic TH-expressing cells, not only where this neurons are, but also in locations of the zefrafish CNS where TH-expressing cells are absent, suggests Mdn can function in a cell-autonomously manner. These results agrees with previous observations that Hairy/E (spl) factors and related genes, generally act cell-autonomously on precursors in the regulation of cell specification (Fischer and Caudy, 1998; Bally-Cuif et al., 2000). Although the action of many transcription factors is likely involved in a given neuronal specification, the present invention indicates that Mdn can function as a single activator of transcription required for the initial cell fate specification of the mesDA cell identity and single bHLH may determine single neuronal cell identity.

Members of the hairy-E (spl) family of bHLH proteins have been shown to be upregulated in vertebrate cells by Notch signalling. An interesting feature found in the third intron of Mdn is the repeat (GTT)₈(ATT)₉, which is characteristic of those genes controlled by Notch. Analysis of the sequences upstream the transcription start site of Mdn, reveals also canonical target sites for Notch (RBP-JK), proneural genes (Box E-Class A), and for E (spl) (Box E-Class B) Taken together, these observations suggest Mdn may also belong to the Notch signalling pathway, suggesting that Mdn can links early patterning events Notch-mediated to the differentiation of defined neuronal precursors into dopaminergic fate. Despite the recent identification of several bHLH hairy-related genes, the unique expression pattern of Mdn suggests a previously unrecognised role for hairy-related genes in mesDA pathway.

Despite of Notch-E (spl) network has been conserved in evolution as a way to assign specific fates to members of groups of initially equivalent cells (Chitnis et al., 1995), we failed to found any ortologue of Mdn when Drosophila cDNA libraries were screened with a Mdn probe. In addition, no matches were found when the nucleotide and amino acid sequences of the bHLH from Mdn was blasted against Drosophila and C. elegans genome sequences, which the entire heterochromatic sequence are already completed. These observations argue in favour of the emerging concept that bHLH proteins can insure the determination of subsets of neuronal phenotypes, and the apparition of new transcription factor genes is causally linked to the appearance of new subclasses of neurons during evolution. Then, Mdn, Hairy and E (spl) genes could originated from the same or closely related ancestral genes, but Mdn has originated later without homologue in Drosophila.

Since the cathelolaminergic system is large involved in human neurodegenerative disorders, p.e. Parkinson's disease, the identification of mouse genes controlling developmental mechanisms of these neurotransmitters, with a special regarding to the mesDA neurons, should provide new insights into the etiology of these disorders. A specific function of bHLH genes in the adult brain is not known, but an emergent concept is that neuronal bHLH proteins are also involved in the “adaptive” changes of mature CNS neurons, an a role in neuronal plasticity has been suggested (Bartholoma and Nave, 1994). Cells from the SVZ act as neural stem cells in both the normal and regenerating brain, and continually generate new neurons destined to the olfactory bulb. Since Mdn is expressed in the SVZ and follows the RMS until the olfactory bulb, we proposed Mdn can be involved also in the differentiation of adult stem cells of the SVZ into dopaminergic neurons, powering the regeneration of such population in the adult brains. Thus, suggesting misexpression of Mdn due to its telomeric position on HC4 can cause a defective regeneration of dopaminergic neurons, and subsequently, a lack of DA neurons in adult brains.

Moreover, by expressing human proteins that specify the formation of specific types of neurons, it may be possible to generate neurons with defined identities. Then, stem cells that have the capacity to self-renew and differentiate into neurons can be cultured and differentiated into DA neurons by expression of MDN.

Therefore, it is an aim of this invention to develop strategies for replacing neurons lost from disease or injury. Since neurodegenerative disorders such Parkinson's disease lead specifically to the loss of DA neurons, an essential aspect of any neural replacement strategy will be the ability to generate DA neurons. With this aim, the function of MDN may exploited, by expressing the gene in embryonic stem cells isolated from humans, to adopt them a DA neuronal fate. Manipulation of stem cells into dopaminergic cells in vitro could be performed in preparation of tissue grafting and experimental therapy for Parkinson's patients (Pogarell and Oertel, 1998; Sautter et al., 1998; Ahlskog, 1993; Defer et al., 1996).

### Bibliography

Ahlskog J E. Cerebral transplantation for Parkinson's disease: current progress and future prospects. Mayo Clin Proc. 1993 June; 68 (6): 578-91

Akazawa C, Sasai Y, Nakanishi S, Kageyama R. Molecular characterization of a rat negative regulator with a basic helix-loop-helix structure predominantly expressed in the developing nervous system. J Biol Chem. 1992 Oct. 25;267 (30): 21879-85.

Altschul S F, Madden T L, Schaffer A A, Zhang J, Zhang Z, Miller W, Lipman D J. Gapped BLAST and PSI-BLAST: a new generation of protein database search programs. Nucleic Acids Res. 1997 Sep. 1;25 (17): 3389-402.

Artavanis-Tsakonas S, Matsuno K, Fortini M E. Notch signaling. Science. 1995 Apr. 14;268 (5208): 225-32.

Bally-Cuif L, Goutel C, Wassef M, Wurst W, Rosa F. Coregulation of anterior and posterior mesendodermal development by a hairy-related transcriptional repressor. Genes Dev. 2000 Jul. 1; 14 (13): 1664-77.

Bartholoma A and Nave K A. NEX-1: a novel brain-specific helix-loop-helix protein with autoregulation and sustained expression in mature cortical neurons. Mech Dev. 1994 December; 48 (3): 217-28.

Blader P, Fischer N, Gradwohl G, Guillemont F, Strahle U. The activity of neurogenini is controlled by local cues in the zebrafish embryo. Development. 1997 November; 124 (22): 4557-69.

Bjorklund, A and Lindvall, O. Catecholaminergic brain stem regulatory systems. In F. E. Blood (ed), Handbook of Physiology, Section 1: The Nervous System, Vol. IV. Bethesda, Md.: American Physiological Society, pp. 155-235. 1984.

Breathnach R, Chambon P. Organization and expression of eucaryotic split genes coding for proteins. Annu Rev Biochem. 1981;50:349-83 Cabrera C V, Alonso M C. Transcriptional activation by heterodimers of the achaete-scute and daughterless gene products of Drosophila. EMBO J. 1991 October; 10 (10): 2965-73.

Campos-Ortega J A (1993) Early neurogenesis in Drosophila melanogaster. In Batze M, Martinez-Arias (eds) The development of Drosophila melanogaster. Cold Spring Harbor Laboratory Press, pp 1091-1129.

Carmena A, Bate M, Jimenez F. Lethal of scute, a proneural gene, participates in the specification of muscle progenitors during Drosophila embryogenesis. Genes Dev. 1995 Oct. 1;9 (19): 2373-83.

Castillo S O, Xiao Q, Lyu M S, Kozak C A, Nikodem V M. Organization, sequence, chromosomal localization, and promoter identification of the mouse orphan nuclear receptor Nurrl gene. Genomics. 1997 Apr. 15;41 (2): 250-7.

Chitnis A, Henrique D, Lewis J, Ish-Horowicz D, Kintner C. Primary neurogenesis in Xenopus embryos regulated by a homologue of the Drosophila neurogenic gene Delta. Nature. 1995 Jun. 29;375 (6534): 761-6.

Corbin V, Michelson A M, Abmayr S M, Neel V, Alcamo E, Maniatis T, Young M W. A role for the Drosophila neurogenic genes in mesoderm differentiation. Cell. 1991 Oct. 18; 67 (2): 311-23.

Dagerlind, A., Friberg, K., Bean, A. J. & Hokfelt, T (1992). Sensitive mRNA detection using unfixed tissue: combined radioactive and non-radioactive in situ hybridization histochemistry. Histochemistry 98, 39-49.

de Celis J F, de Celis J, Ligoxygakis P, Preiss A, Delidakis C, Bray S. Functional relationships between Notch, Su (H) and the bHLH genes of the E (spl) complex: the E (spl) genes mediate only a subset of Notch activities during imaginal development. Development. 1996 September; 122 (9): 2719-28.

Defer G L, Geny C, Ricolfi F, Fenelon G, Monfort J C, Remy P, Villafane G, Jeny R, Samson Y, Keravel Y, Gaston A, Degos J D, Peschanski M, Cesaro P, Nguyen J P. Long-term outcome of unilaterally transplanted parkinsonian patients. I. Clinical approach. Brain. 1996 February; 119 (Pt 1): 41-50.

de la Pompa J L, Wakeham A, Correia K M, Samper E, Brown S, Aguilera R J, Nakano T, Honjo T, Mak T W, Rossant J, Conlon R A. Conservation of the Notch signalling pathway in mammalian neurogenesis. Development. 1997 March; 124 (6): 1139-48

Delidakis C, Artavanis-Tsakonas S. The Enhancer of split [E (spl)] locus of Drosophila encodes seven independent helix-loop-helix proteins. Proc Natl Acad Sci USA. 1992 Sep. 15;89 (18): 8731-5.

Doetsch F, Alvarez-Buylla A. Network of tangential pathways for neuronal migration in adult mammalian brain. Proc Natl Acad Sci USA. 1996 Dec. 10;93 (25): 14895-900.

Echelard Y, Epstein D J, St-Jacques B, Shen L, Mohler J, McMahon JA, McMahon AP. Sonic hedgehog, a member of a family of putative signalling molecules, is implicated in the regulation of CNS polarity. Cell. 1993 Dec. 31;75 (7): 1417-30.

Ericson J, Muhr J, Jessell T M, Edlund T. Sonic hedgehog: a common signal for ventral patterning along the rostrocaudal axis of the neural tube. Int J Dev Biol. 1995 October; 39 (5): 809-16. Review.

Fisher A, Caudy M. The function of hairy-related bHLH repressor proteins in cell fate decisions. Bioessays. 1998 April; 20 (4): 298-306.

Forno L S. Neuropathologic features of Parkinson's, Huntington's, and Alzheimer's diseases. Ann N Y Acad Sci. 1992 May 11;648:6-16.

Frohman, M. (1993) Rapid amplification of complementary DNA ends for Generation of full-length complementary DNAs: Thermal RACE. Methods Enzymol. 218, 340-356.

Garrell J and Campuzano S. The helix-loop-helix domain: a common motif for bristles, muscles and sex. Bioessays. 1991 October; 13 (10): 493-8.

Golbe L I. The genetics of Parkinson's disease. Rev Neurosci. 1993 Jan-Mar; 4 (1): 1-16.

Graham F L, Smiley J, Russell W C, Nairn R. Characteristics of a human cell line transformed by DNA from human adenovirus type 5. J Gen Virol. 1977 July; 36 (1): 59-74).

Graham F L, Eb A J van der. A new technique for the assay of infectivity of human adenovirus 5 DNA. Virology. 1973 April; 52 (2): 456-67.

Grace A A, Bunney B S, Moore H, Todd C L. Dopamine-cell depolarization block as a model for the therapeutic actions of antipsychotic drugs. Trends Neurosci 1997 January; 20 (1): 31-7.

Gubler U, Hoffman B J. A simple and very efficient method for generating cDNA libraries. Gene. 1983 November; 25 (2-3): 263-9.

Hauptmann G, Gerster T. Two-color whole-mount in situ hybridization to vertebrate and Drosophila embryos. Trends Genet. 1994 August; 10 (8): 266.

Hynes M and Rosenthal A. Specification of dopaminergic and serotonergic neurons in the vertebrate CNS. Curr Opin Neurobiol. 1999 February; 9 (1): 26-36.

Jan Y N and Jan L Y. HLH proteins, fly neurogenesis, and vertebrate myogenesis. Cell. 1993 Dec. 3;75 (5): 827-30.

Jarriault S, Brou C, Logeat F, Schroeter E H, Kopan R, Israel A. Signalling downstream of activated mammalian Notch. Nature. 1995 Sep. 28;377 (6547): 355-8.

Jellinger K. Morphology and biochemistry of Parkinson's syndrome]. Cesk Patol. 1973 February; 9 (1): 1-13.

Jennings B, Preiss A, Delidakis C, Bray S. The Notch signalling pathway is required for Enhancer of split bHLH protein expression during neurogenesis in the Drosophila embryo. Development. 1994 December; 120 (12): 3537-48.

Klambt C, Knust E, Tietze K, Campos-Ortega J A. Closely related transcripts encoded by the neurogenic gene complex enhancer of split of Drosophila melanogaster. EMBO J. 1989 January; 8 (1): 203-10.

Knust E, Schrons H, Grawe F, Campos-Ortega J A. Seven genes of the Enhancer of split complex of Drosophila melanogaster encode helix-loop-helix proteins. Genetics. 1992 October; 132 (2): 505-18.

Lee J E. Basic helix-loop-helix genes in neural development. Curr Opin Neurobiol. 1997 February; 7 (1): 13-20.

Lois C, Alvarez-Buylla A. Long-distance neuronal migration in the adult mammalian brain. Science. 1994 May 20;264 (5162): 1145-8.

Lumsden A, Krumlauf R. Patterning the vertebrate neuraxis. Science. 1996 Nov. 15;274 (5290): 1109-15.

Luskin M B. Restricted proliferation and migration of postnatally generated neurons derived from the forebrain subventricular zone. Neuron. 1993 July; 11 (1): 173-89.

Marchuk D, Drumm M, Saulino A, Collins F S. Construction of T-vectors, a rapid and general system for direct cloning of unmodified PCR products. Nucleic Acids Res. 1991 Mar 111;19 (5): 1154.

Martinez C, Modolell J, Garrell J. Regulation of the proneural gene achaete by helix-loop-helix proteins. Mol Cell Biol. 1993 June; 13 (6): 3514-21.

Müller, M., v. Weizsäcker, E., and Campos-Ortega, J. A. 1996. Transcription of a zebrafish gene of the hairyEnhancer of split family delineates the midbrain anlage in the neural plate. Dev. Genes Evol. 206: 153-160.

Murre C, Bain G, van Dijk M A, Engel I, Furnari B A, Massari M E, Matthews J R, Quong M W, Rivera R R, Stuiver M H. Structure and function of helix-loop-helix proteins. Biochim Biophys Acta. 1994 Jun. 21;1218 (2): 129-35.

Nagatsu T, Levitt M, Udenfriend S. Conversion of L-tyrosine to 3,4-dihydroxyphenylalanine by cell-free preparations of brain and sympathetically innervated tissues. Biochem Biophys Res Commun. 1964;14:543-9.

Nakagawa O, Nakagawa M, Richardson J A, Olson E N, Srivastava D. HRT1, HRT2, and HRT3: a new subclass of bHLH transcription factors marking specific cardiac, somitic, and pharyngeal arch segments. Dev Biol. 1999 Dec. 1;216 (1): 72-84.

Nissl, F, (1894). Ueber die sogenannten Granula der Nervenzellen. Neurolog. Centralblatt 13, 781-789.

Olson E N. MyoD family: a paradigm for development? Genes Dev. 1990 September; 4 (9): 1454-61

Pogarell O and Oertel W H. Neural transplantation in Parkinson's disease and its effects on rest tremor: a review of the literature. Mov Disord. 1998;13 Suppl 3:101-2

Ritz M C, Lamb R J, Goldberg S R, Kuhar M J. Cocaine receptors on dopamine transporters are related to selfadministration of cocaine. Science. 1987 Sep. 4;237 (4819): 1219-23.

Rossner M J, Dorr J, Gass P, Schwab M H, Nave K A. SHARPs: mammalian enhancer-of-split- and hairy-related proteins coupled to neuronal stimulation. Mol Cell Neurosci. 1997;10 (3-4): 460-75.

Ruohola H, Bremer K A, Baker D, Swedlow J R, Jan L Y, Jan Y N. Role of neurogenic genes in establishment of follicle cell fate and oocyte polarity during oogenesis in Drosophila. Cell. 1991 Aug. 9;66 (3): 433-49.

Rushlow C A, Hogan A, Pinchin S M, Howe K M, Lardelli M, Ish-Horowicz D. The Drosophila hairy protein acts in both segmentation and bristle patterning and shows homology to N-myc. EMBO J. 1989 October; 8 (10): 3095-103.

Sasai Y, Kageyama R, Tagawa Y, Shigemoto R, Nakanishi S. Two mammalian helix-loop-helix factors structurally related to Drosophila hairy and Enhancer of split. Genes Dev. 1992 December; 6 (12B): 2620-34.

Sautter J, Meyer M, Spenger C, Seiler R W, Widmer H R. Effects of combined BDNF and GDNF treatment on cultured dopaminergic midbrain neurons. Neuroreport. 1998 Apr. 20;9 (6): 1093-6

Seeman P, Guan H C, Van Tol H H. Dopamine D4 receptors elevated in schizophrenia. Nature. 1993 Sep. 30;365 (6445): 441-5.

Shapiro M B, Senapathy P. RNA splice junctions of different classes of eukaryotes: sequence statistics and functional implications in gene expression. Nucleic Acids Res. 1987 Sep. 11;15 (17): 7155-74.

Shen M, Kawamoto T, Yan W, Nakamasu K, Tamagami M, Koyano Y, Noshiro M, Kato Y. Molecular characterization of the novel basic helix-loop-helix protein DEC1 expressed in differentiated human embryo chondrocytes. Biochem Biophys Res Commun. 1997 Jul. 18;236 (2): 294-8.

Smeets, W J. A. J., and Reiner, A. “Phylogeny and development of Catecholamine System in the CNS of Vertebrates”. Cambridge Univ. Press, Cambridge, UK. 1994.

Smidt M P, van Schaick H S, Lanctot C, Tremblay J J, Cox J J, van der Kleij A A, Wolterink G, Drouin J, Burbach J P. A homeodomain gene Ptx3 has highly restricted brain expression in mesencephalic dopaminergic neurons. Proc Natl Acad Sci USA. 1997 Nov. 25;94 (24): 13305-10.

Specht L A, Pickel V M, Joh T H, Reis D J. Light-microscopic immunocytochemical localization of tyrosine hydroxylase in prenatal rat brain. II. Late ontogeny. J Comp Neurol. 1981 Jun. 20;199 (2): 255-76.

Sporle R, Schughart K. Paradox segmentation along inter- and intrasomitic borderlines is followed by dysmorphology of the axial skeleton in the open brain (opb) mouse mutant. Dev Genet. 1998;22 (4): 359-73.

Temple S and Alvarez-Buylla A. Stem cells in the adult mammalian central nervous system. Curr Opin Neurobiol. 1999 February; 9 (1): 135-41.

Van Doren M, Powell P A, Pasternak D, Singson A, Posakony J W. Spatial regulation of proneural gene activity: auto- and cross-activation of achaete is antagonized by extramacrochaetae. Genes Dev. 1992 December; 6 (12B): 2592-605.

Voorn P, Kalsbeek A, Jorritsma-Byham B, Groenewegen H J. The pre- and postnatal development of the dopaminergic cell groups in the ventral mesencephalon and the dopaminergic innervation of the striatum of the rat. Neuroscience. 1988 June; 25 (3): 857-87.

Xu T, Caron L A, Fehon R G, Artavanis-Tsakonas S. The involvement of the Notch locus in Drosophila oogenesis. Development. 1992 August; 115 (4): 913-22.

Ye W, Shimamura K, Rubenstein J L, Hynes M A, Rosenthal A. FGF and Shh signals control dopaminergic and serotonergic cell fate in the anterior neural plate. Cell. 1998 May 29;93 (5): 755-66.

Further literature, to which reference is made in the description by the respective numbers:

1. Derossi D, Chassaing G, Prochiantz A: Trojan peptides: the penetratin system for intracellular delivery. Trends Cell Biol 1998, 8:84-87

2. Lindgren M, Hüllbrink M, Prochiantz A, Langel U: Cell-penetrating peptides. Trends Pharmacol Sci 2000, 21:99-103

3. Schwarze S R, Dowdy S F: In vivo protein transduction: intracellular delivery of biologically active protein, compounds and DNA. Trends Pharmacol Sci 2000, 21:45-48

4. Schwarze S R, Ho A, Vocero-Akbani A, Dowdy S F: In vivo protein transduction: delivery of a biologically active protein into the mouse. Science 1999, 285:1569-1572

5. Rousselle C, Clair P, Lefauconnier J-M, Kackzorek M, Scherrmann J-M, Temsamani J: New advances in the transport of doxorubicin through the blood brain barrier by a peptide vector mediated strategy. Mol Pharmacol 2000, 57:679-686

6. Prochiantz A. Messenger proteins: homeoproteins, TAT and others. Curr Opin Cell Biol. 2000 August; 12 (4): 400-6

7. Lin Y Z, Yao S Y, Veach R A, Torgerson T R, Hawiger J. Inhibition of nuclear translocation of transcription factor NF-kappa B by a synthetic peptide containing a cell membrane-permeable motif and nuclear localization sequence. J Biol Chem. 1995 Jun. 16;270 (24): 14255-8.)

8. Schwarze S R, Hruska K A, Dowdy S F. Protein transduction: unrestricted delivery into all cells? Trends Cell Biol. 2000 July; 10 (7): 290-5

9. Frankel A D, Pabo C O. Cellular uptake of the tat protein from human immunodeficiency virus. Cell. 1988 Dec. 23;55 (6): 1189-93

10. Green M, Loewenstein P M. Autonomous functional domains of chemically synthesized human immunodeficiency virus tat trans-activator protein. Cell. 1988 Dec. 23;55 (6): 1179-88.)

11. Vives E, Brodin P, Lebleu B: A truncated HIV-1 Tat protein basic domain translocates through the plasma membrane and accumulates in the cell nucleus. J Biol Chem 1997, 272:16010-16017

12. Schwarze S R, Ho A, Vocero-Akbani A, Dowdy S F. In vivo protein transduction: delivery of a biologically active protein into the mouse. Science. 1999 Sep. 3;285 (5433): 1569-72

TABLE 1
PCR Primers Pairs for MDN mutation analysis

| | | | | |
|---|---|---|---|---|
| FORWARD PRIMER NAME | REVERSE PRIMER NAME | Size | Tm | Exon |
| sequence (5′ to 3′) | sequence (5′ to 3′) | (bp) | (° C.) | amplified |

| | | | | | | |
|---|---|---|---|---|---|---|
| MDN1D | ctgatcttgaatgcatacatcc | MDN1R | cgggtcggtgagtcagatgc | 311 | 56 | 1 |
| MDN2D | cccttcctagagcgaatctgag | MDN2R | gggcgtctccgcagagtgg | 301 | 56 | 2 |
| MDN3D | gcagggcgaacctcaggag | MDN3R | ctcgggaacactcagtcactcc | 318 | 56 | 3 |
| MDN4aD | gtgccctgcacccctttgg | MDN4aR | aggcggccaggggaaagg | 392 | 56 | 4^{a} |
| MDN4bD | ggcgaggccgctgtgttcc | MDN4bR | ggagatccttcagaagactc | 392 | 56 | 4^{b} |

^{a}Exon 4 is too large for SSCA analysis. Thus, primers were designed to amplify shorter fragments covering the coding region.

What is claimed is:

1. A purified and isolated DNA encoding a mammalian bHLH transcription factor for the induction of neural cells.

2. The DNA of claim 1, encoding a mouse bHLH transcription factor.

3. The DNA of claim 1, which is a cDNA comprising the sequence shown in Seq. ID. No. 1 or a portion thereof, which encodes a biologically active transcription factor.

4. The DNA of claim 1, which comprises the nucleotide sequence from nucleotide 145 through 252 of Seq. ID. No. 1.

5. The DNA of claim 1, which is a genomic DNA comprising the sequence shown in Seq. ID. No. 3 or a portion thereof, which encodes a biologically active transcription factor.

6. The DNA of claim 1, encoding a human bHLH transcription factor.

7. The DNA of claim 1, which is a cDNA sequence comprising the sequence shown in Seq. ID. No. 2 or a portion thereof, which encodes a biologically active transcription factor.

8. The DNA of claim 1, which is a genomic DNA comprising the sequence shown in Seq. ID. No. 4 or a portion thereof, which encodes a biologically active transcription factor.

9. An isolated nucleotide which comprises the complement of any one of the nucleotides of claim 1.

10. Purified isolated mammalian transcription factor for the induction of neural cells encoded by the DNA of claim 1 or variants thereof, provided that said variants comprise nucleic acid changes due to the degeneracy of the genetic code, which code for the same or functionally equivalent transcription factor as the nucleic acid of claim 1 or provided that said variants hybridize under stringent conditions to a nucleic acid which comprises the sequence of claim 1 and further provided that said variants code for a protein with activity as transcription factor for the induction of neural cells.

11. Purified isolated mouse transcription factor for the induction of neural cells comprising the amino acid of Seq. ID. No. 5 and homologues or fragments thereof which retain biological activity.

12. Purified isolated human transcription factor for the induction of neural cells comprising the amino acid of Seq. ID. No. 6 and homologues or fragments thereof which retain biological activity.

13. A fusion protein, comprising the transcription factor of claim 10 fused to a signal peptide, which allows the delivery of said transcription factor into a target cell.

14. The fusion protein of claim 13, wherein the signal peptide is the HIV-1 TAT sequence and optionally further comprises a His-tag and/or an epitope tag.

15. An expression vector comprising the DNA of claim 1 or a DNA, which codes for the fusion protein of claim 13 or 14.

16. A host cell transformed with the vector of claim 15.

17. The host cell of claim 16, which is a vertebrate stem cell.

18. The host cell of claim 17, which is a mammalian stem cell.

19. The host cell of claim 18, which is a mouse stem cell.

20. The host cell of claim 19, which is a human stem cell.

21. The host cell of claim 20, which is an embryonic stem cell.

22. The host cell of claim 20, which is an adult stem cell.

23. A method for producing the transcription factor of claim 10 in a substantially pure form, which comprises transforming a host cell of claim 16 with the vector of claim 15, culturing the host cell under conditions which permit expression of the sequence by the host cell and isolating the peptide from the host cell.

24. An antibody which specifically binds to the protein of claim 10.

25. The antibody of claim 24, wherein said antibody is selected from the the group consisting of a polyclonal antibody, a monoclonal antibody, a humanized antibody, a chimeric antibody, and a synthetic antibody.

26. The antibody of claims 24 and 25 wherein said antibodies are linked to a chemotherapeutic agent or toxic agent and/or to an imaging agent.

27. A hybridoma which produces a monoclonal antibody having binding specificity to any one of the proteins of claim 10.

28. A recombinant non-human vertebrate in which the DNA of claim 1 has been inactivated.

29. A recombinant mouse, in which the DNA of claim 5 has been inactivated.

30. A nucleic acid probe comprising a nucleic acid sequence complementary to any one of the nucleic acid sequences of claim 1 or a portion thereof.

31. A test kit, comprising the probes of claim 30 and means for detecting or measuring the hybridization of said probes to the sequences comprised of claim 1.

32. An ex vivo method of producing dopaminergic neurons, which comprises the following steps:
a) providing neural embryonic stem cells, neural adult stem cells and/or embryonic stem cells;
b) contacting said cells with an effective amount of the transcription factor of claim 10;
c) culturing said cells under conditions, which allow the specification and differentiation to dopaminergic neurons; and
d) recovering the dopaminergic neurons.

33. Dopaminergic neurons, which are obtainable by the method of claim 32.

34. A composition, which comprises an effective amount of the dopaminergic neurons of claim 33 in combination with a pharmaceutically acceptable carrier.

35. A composition comprising an effective amount of a protein of claim 10, in combination with a pharmaceutically acceptable carrier.

36. A composition comprising nucleic acid sequences of claim 1.

37. A composition comprising a host cell of claim 16.

38. A composition comprising the antibody of claim 24.

39. Use of the composition of claim 38 in the in vitro or in vivo diagnosis of neurodegenerative disorders.

40. A method of treating a patient suffering from a neurodegenerative disease comprising administering an effective amount of the composition of claim 34, 35 or 36 to said patient, thereby substituting degenerated or lost nerval cells in said patient.

41. The method of claim 40, wherein the compositions are administered intracerebrally.

42. The method of claim 40, wherein the compositions are administered intraperitoneally.

43. The method of claim 40, wherein the neurodegenerative disease is Parkinson's disease.

44. The method of claim 40, wherein a vertebrate is treated.

45. The method of claim 40, wherein a mammal, preferably a human patient is treated.
